(19) United States
(12) Patent Application Publication
(10) Pub. No.: US 2024/0358327 A1
(43) Pub. Date: Oct. 31, 2024

(54) INTRA-ORAL DEVICES AND METHODS FOR MAKING AND USING THEM

(71) Applicant: ProSomnus Sleep Technologies, Inc., Pleasanton, CA (US)

(72) Inventors: Sung KIM, Pleasanton, CA (US); Leonard A. Liptak, Pleasanton, CA (US); Brittany Logan Austin, Walnut Creek, CA (US)

(21) Appl. No.: 18/646,747

(22) Filed: Apr. 25, 2024

Related U.S. Application Data
(60) Provisional application No. 63/461,694, filed on Apr. 25, 2023.

(51) Int. Cl. A61B 5/00; A61B 5/024; A61B 5/0295

(52) U.S. Cl. CPC A61B 5/682; A61B 5/02427; A61B 5/0295; A61B 2560/0406; A61B 2560/045; A61B 2560/0462; A61B 2562/0257; A61B 2562/164; A61B 2562/166

(57) ABSTRACT

A device is disclosed that includes a device body configured to couple to dentition. The device may include a sensor configured to couple to the device body to be positioned to be adjacent to soft tissue or hard tissue in an oral cavity when the device body couples to the dentition.

### CROSS-REFERENCE TO RELATED APPLICATIONS

This application claims priority to and the benefit of U.S. Provisional Patent Application Ser. No. 63/461,694, filed Apr. 25, 2023, and entitled “INTRA-ORAL DEVICES AND METHODS FOR MAKING AND USING THEM”, the disclosure of which is incorporated herein by reference in its entirety.

### TECHNICAL FIELD

This invention generally relates to dental devices and related methods for making and using.

### BACKGROUND

Dental devices such as orthodontic devices and mandibular advancement devices (MAD) have a long residence time inside a patient's mouth. For example, for a MAD, the intra-oral dwell time is throughout the time the patient is asleep. During this time, many physiological and physical changes occur in the patient's body that affect the efficacy of the MAD and the patient's health.

### SUMMARY

This Summary is provided to introduce a selection of concepts in simplified form that can be further described below in the Detailed Description. This Summary is not intended to identify key aspects or essential aspects of the claimed subject matter. All features of exemplary embodiments which can be described in this disclosure and can be not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with any accompanying Figures.

The present disclosure relates to a device including: a device body configured to couple to dentition; and a sensor configured to couple to the device body to be positioned to be adjacent to soft tissue or hard tissue in an oral cavity when the device body couples to the dentition.

The present disclosure relates to a device, wherein the sensor is configured to obtain information from the adjacent soft tissue or the adjacent hard tissue when the device body couples to the dentition.

The present disclosure relates to a device, further including a device configured to be operably linked to or connected to the sensor to receive the obtained information.

The present disclosure relates to a device, wherein the device is disposed outside the oral cavity.

The present disclosure relates to a device, wherein the sensor is configured to be coupled to the device to be at least partially exposed to an outer surface of the device.

The present disclosure relates to a device, wherein the sensor is configured to contact or be positioned to be close to the soft tissue or the hard tissue in the oral cavity.

The present disclosure relates to a device, wherein the sensor is a flexible sensor.

The present disclosure relates to a device, wherein the sensor is not expandable or stretchable.

The present disclosure relates to a device, wherein the device includes a recess or a protrusion on a surface of the device, and wherein the sensor is configured to be coupled to the recess or the protrusion.

The present disclosure relates to a device, wherein the recess include the protrusion to push the sensor against the soft tissue or the hard tissue.

The present disclosure relates to a device, wherein the recess or the protrusion includes a slot, a groove, or an insert, and wherein the sensor is configured to be coupled to the slot, the groove, or the insert.

The present disclosure relates to a device, wherein the device includes a curved surface, wherein the sensor is an inflexible sensor, or a flat sensor, or a non-curved sensor, wherein the sensor is positioned in the device with respect to the curved surface of the device.

The present disclosure relates to a device, wherein the curved surface of the device corresponds to a curve of a dental arch in the oral cavity.

The present disclosure relates to a device, wherein the device includes data storage operably linked to or connected to the sensor.

The present disclosure relates to a device, wherein the data storage is built into a Radio Frequency Identity (RFID) chip or Near-Field Communication (NFC) chip.

The present disclosure relates to a device, wherein the recess or the protrusion has a buccal lingual thickness from about 0.5 to about 15 mm deep.

The present disclosure relates to a device, wherein a maximum distance between a surface of the sensor facing toward the soft tissue or the hard tissue and an outer surface of the device is from about 0.5 mm to about 5 mm.

The present disclosure relates to a device, wherein the sensor is a photoplethysmography (PPG) sensor, a physiological sensor, a physical sensor, a chemical sensor, a proximity sensor, a vibration sensor, or a positional sensor.

The present disclosure relates to a device, wherein the sensor includes a silicone, plastic, a liquid crystal polymer (LCP) substrate, an LCP base material adhered to a cladding, a fiber or a yarn, paper (optionally including cellulose nanofibers (CNFs), a polyaniline nanofiber/graphite nanofiber (PANI/GNF) nanocomposite, or a polyaniline (PANI) supportive matrix, or a combination thereof.

The present disclosure relates to a device, wherein the device is a mandibular advancement device (MAD), or an orthodontic device, or a device that repositions a mandible or a maxilla.

The details of one or more exemplary embodiments of the invention are set forth in the accompanying drawings and the description below. Other features, objects, and advantages of the invention will be apparent from the description and drawings, and from the claims.

All publications, patents, patent applications, cited herein are hereby expressly incorporated by reference in their entireties for all purposes.

### DESCRIPTION OF DRAWINGS

It is to be expressly understood that the drawings set forth herein are illustrative of exemplary embodiments provided herein and are not meant to limit the scope of the invention as encompassed by the claims.

FIG. 1 illustrates a PPG sensor that is mounted in or on a flexible circuit board for a mandibular advancement device in some embodiments.

FIG. 2 illustrates flexible circuit board configurations for two different arch forms with changed radiuses of the slot cut to allow insertion of a flexible sensor circuit board in some embodiments.

FIG. 3 illustrates example structures with added extra thickness to create more spring force to teeth which need torquing and rotating in some embodiments.

FIG. 4 illustrates a flexible circuit board with the sensor or sensors placed in a groove or inset cut or slot, or otherwise designed into either/or the outer surface of a product of manufacture in some embodiments.

FIG. 5 illustrates an orthodontic device with a sensor pocket and a circuit channel design in some embodiments.

FIG. 6 illustrates an orthodontic device with a sensor pocket and bend points in some embodiments.

FIG. 7 illustrates a sensor design with bend points orthodontic device in some embodiments.

### DETAILED DESCRIPTION

Dental devices such as orthodontic devices and mandibular advancement devices (MAD) have a long residence time inside a patient's mouth. For example, for a MAD, the intra-oral dwell time is throughout the time the patient is asleep. During this time, many physiological and physical changes occur in the patient's body that affect the efficacy of the MAD and the patient's health.

In some embodiments, it can be useful to allow the MAD respond in real time to the changes in the patient in order to provide the most effective mandibular position adjustment for the patient at the particular time. In addition, physicians may benefit from knowing both real time changes in the patient's body and the history of these changes while the dental device, for example, a MAD, is being used in order to provide a better treatment regimen or react to a real time medical issue. Accordingly, means to accurately measure body physiological and physical changes by the dental devices are provided.

The present disclosure relates to a device including: a device body configured to couple to dentition; and a sensor configured to couple to the device body to be positioned to be adjacent to soft tissue or hard tissue in an oral cavity when the device body couples to the dentition.

The present disclosure relates to a device, wherein the sensor is configured to obtain information from the adjacent soft tissue or the adjacent hard tissue when the device body couples to the dentition.

The present disclosure relates to a device, further including a device configured to be operably linked to or connected to the sensor to receive the obtained information.

The present disclosure relates to a device, wherein the device is disposed outside the oral cavity.

The present disclosure relates to a device, wherein the sensor is configured to be coupled to the device to be at least partially exposed to an outer surface of the device.

The present disclosure relates to a device, wherein the sensor is configured to contact or be positioned to be close to the soft tissue or the hard tissue in the oral cavity.

The present disclosure relates to a device, wherein the sensor is a flexible sensor.

The present disclosure relates to a device, wherein the sensor is not expandable or stretchable.

The present disclosure relates to a device, wherein the device includes a recess or a protrusion on a surface of the device, and wherein the sensor is configured to be coupled to the recess or the protrusion.

The present disclosure relates to a device, wherein the recess include the protrusion to push the sensor against the soft tissue or the hard tissue.

The present disclosure relates to a device, wherein the recess or the protrusion includes a slot, a groove, or an insert, and wherein the sensor is configured to be coupled to the slot, the groove, or the insert.

The present disclosure relates to a device, wherein the device includes a curved surface, wherein the sensor is an inflexible sensor, or a flat sensor, or a non-curved sensor, wherein the sensor is positioned in the device with respect to the curved surface of the device.

The present disclosure relates to a device, wherein the curved surface of the device corresponds to a curve of a dental arch in the oral cavity.

The present disclosure relates to a device, wherein the device includes data storage operably linked to or connected to the sensor.

The present disclosure relates to a device, wherein the data storage is built into a Radio Frequency Identity (RFID) chip or Near-Field Communication (NFC) chip.

The present disclosure relates to a device, wherein the recess or the protrusion has a buccal lingual thickness from about 0.5 to about 15 mm deep.

The present disclosure relates to a device, wherein a maximum distance between a surface of the sensor facing toward the soft tissue or the hard tissue and an outer surface of the device is from about 0.5 mm to about 5 mm.

The present disclosure relates to a device, wherein the sensor is a photoplethysmography (PPG) sensor, a physiological sensor, a physical sensor, a chemical sensor, a proximity sensor, a vibration sensor, or a positional sensor.

The present disclosure relates to a device, wherein the sensor includes a silicone, plastic, a liquid crystal polymer (LCP) substrate, an LCP base material adhered to a cladding, a fiber or a yarn, paper (optionally including cellulose nanofibers (CNFs), a polyaniline nanofiber/graphite nanofiber (PANI/GNF) nanocomposite, or a polyaniline (PANI) supportive matrix, or a combination thereof.

The present disclosure relates to a device, wherein the device is a mandibular advancement device (MAD), or an orthodontic device, or a device that repositions a mandible or a maxilla.

In some embodiments, provided are products of manufacture, also referred to herein as intra-oral or dental devices, that have flexible sensors that are configured to follow the contours of a patient's hard or soft tissue.

In some embodiments, an intra-oral device is provided, which may include a device body including an engagement member to engage a dentition of a patient, a sensor coupled to the device body to be positioned to be adjacent to soft tissue or hard tissue in an oral cavity of the patient when the intra-oral device is positioned or placed in the oral cavity of the patient, and a control device operably linked to or connected to the sensor.

In some embodiments, the present disclosure relates to an intra-oral device, wherein the device is disposed outside the oral cavity.

In some embodiments, the present disclosure relates to an intra-oral device, wherein the sensor is to be coupled to the body to be at least partially exposed to an outer surface of the body.

In some embodiments, the present disclosure relates to an intra-oral device, wherein the sensor is to contact the soft tissue or the hard tissue in the oral cavity.

In some embodiments, the present disclosure relates to an intra-oral device, wherein the sensor is a flexible sensor. In some embodiments, the present disclosure relates to an intra-oral device, wherein the sensor is not expandable or stretchable

In some embodiments, the present disclosure relates to an intra-oral device, wherein the device body include a slot, a groove, or an insert on a surface of the device body, and wherein the sensor is to be coupled to the slot, the groove, or the insert of the device.

In some embodiments, the present disclosure relates to an intra-oral device, wherein the device body includes a curved surface, wherein the sensor is an inflexible, or flat, or non-curved sensor, wherein the sensor is positioned in the device body with respect to the curved surface of the intra-oral device.

In some embodiments, the present disclosure relates to an intra-oral device, wherein the curve of the curved surface of the intra-oral device corresponds to a curve of a dental arch in the oral cavity.

In some embodiments, the present disclosure relates to an intra-oral device, wherein the control device includes data storage operably linked to or connected to the sensor.

In some embodiments, the present disclosure relates to an intra-oral device, wherein the data storage is built into a Radio Frequency Identity (RFID) chip or Near-Field Communication (NFC) chip.

In some embodiments, the present disclosure relates to an intra-oral device, wherein the slot, the groove or the insert is between about 0.5 to about 15 mm deep.

In some embodiments, the present disclosure relates to an intra-oral device, wherein a maximum length from the sensor to an outer surface of the device body is between about 0.5 mm to about 5 mm.

In some embodiments, the present disclosure relates to an intra-oral device, wherein the sensor can be a photoplethysmography (PPG) sensor, a physiological sensor, a physical sensor, a chemical sensor, a proximity sensor, a vibration sensor, a positional sensor, or any other similar sensor.

In some embodiments, the present disclosure relates to an intra-oral device, wherein the sensor includes a silicone, plastic (optionally, an acetate), a liquid crystal polymer (LCP) substrate, an LCP base material adhered to a cladding (optionally a thin copper cladding), a fiber or a yarn, paper (optionally including cellulose nanofibers (CNFs), a polyaniline nanofiber/graphite nanofiber (PANI/GNF) nanocomposite, or a polyaniline (PANI) supportive matrix, or a combination thereof.

The present disclosure relates to an intra-oral device, wherein the intra-oral device can be a mandibular advancement device (MAD), an orthodontic device, or an intra-oral device designed to reposition the mandible or the maxilla.

In some embodiments, provided are manufactured products, intra-oral or dental devices, which can feature an inflexible, flat, or non-curved sensor situated on a curved surface of the intra-oral device or within a slot, groove, or inset on its surface. Optionally, the curvature of the intra-oral device's surface closely mirrors that of a dental arch.

In some embodiments, manufactured products, intra-oral or dental devices, such as orthodontic devices and mandibular advancement devices (MADs), incorporate flexible sensors positioned on the device's surface to closely conform to the curved contour of a patient's hard or soft tissues. This close contact enables the flexible sensor to provide more accurate readings of the patient's physiological state.

In some embodiments, photoplethysmography (PPG) sensors may be employed in manufactured products, intra-oral or dental devices, as described herein, to measure volumetric variations in blood circulation. Since PPG sensors utilize optical measurements to monitor blood circulation and heart rate, having the PPG sensor conform to the contour of a patient's soft tissues enhances the accuracy of the measurements.

In some embodiments, utilizing a PPG mounted on a flexible sensor can enable the precise measurement of physiological data.

In some embodiments, manufactured products provided in certain embodiments include intra-oral or dental devices. These products may feature a flexible sensor positioned on, adhered to, or implanted in the outer surface of the intra-oral device, ensuring close contact with a patient's soft or hard tissues (e.g., tooth structure) when the device is placed in the patient's mouth or oral cavity. The flexible sensor, though flexible, may have fixed dimensions and may be located within a slot, groove, or inset on the device's surface. Additionally, the device's surface curvature may align with that of a dental arch.

Some embodiments may incorporate a data storage capability or unit linked to the flexible sensor, with options including a data storage feature integrated into a Radio Frequency Identity (RFID) chip or Near-Field Communication (NFC) chip.

In some embodiments, the slot, groove, or insert included in the device may have a depth ranging from about 0.5 to about 15 mm, for example, about 1 mm, about 2 mm, about 3 mm, about 4 mm, about 5 mm, about 6 mm, about 7 mm, about 8 mm, about 9 mm, or about 10 mm. Additionally, in some embodiments, in manufactured products, the maximum distance from the sensor (such as a flexible sensor or an inflexible, flat, or non-curved sensor) to the outer surface or soft or hard tissue of the patient can be between about 0.5 mm to about 5 mm, e.g., less than about 1 mm, resulting in more precise physiological measurements.

In some embodiments, the sensor, whether flexible or inflexible, may encompass various types, functions, and measurement capabilities, including photoplethysmography (PPG) sensors, physiological sensors, physical sensors, chemical sensors, proximity sensors, vibration sensors, positional sensors, or combinations thereof.

In some embodiments, materials for manufacturing the sensor, whether flexible or inflexible, may include silicone, plastic (possibly acetate), liquid crystal polymer (LCP) substrate, LCP base material with cladding (possibly thin copper cladding), fiber or yarn, paper (potentially containing cellulose nanofibers (CNFs), polyaniline nanofiber/graphite nanofiber (PANI/GNF) nanocomposite, polyaniline (PANI) supportive matrix deposited on an optical fiber core acting as an active cladding), vinylidene fluoride-trifluoroethylene-hexafluoroacetone copolymer, or combinations thereof.

Some embodiments may involve manufactured products or intra-oral devices functioning as mandibular advancement devices (MADs), orthodontic devices, or intra-oral devices repositioning the mandible or maxilla.

In some embodiments, the present disclosure relates to an intra-oral device, wherein the product of manufacture or the intra-oral device is a mandibular advancement device (MAD), or an orthodontic device, or an intra-oral device that repositions the mandible or the maxilla.

In some embodiments, provided are products of manufacture, or intra-oral or dental devices, having an inflexible, or flat, or non-curved sensor positioned on a curved surface of the intra-oral device, or positioned in or substantially within a slot, groove or inset, on a surface of the intra-oral device, and optionally the curve of the surface of the intra-oral device substantially follows the curve of a dental arch.

The present disclosure relates to an intra-oral device including: a device body configured to couple to dentition, and a sensor configured to couple to the device body to be positioned to be adjacent to soft tissue or hard tissue in an oral cavity when the device body couples to the dentition.

The present disclosure relates to an intra-oral device, wherein the sensor is configured to obtain information from the adjacent soft tissue or the adjacent hard tissue when the device body couples to the dentition.

The present disclosure relates to an intra-oral device, further including a device configured to be operably linked to or connected to the sensor to receive the obtained information.

The present disclosure relates to an intra-oral device, wherein the device is disposed outside the oral cavity.

The present disclosure relates to an intra-oral device, wherein the sensor is configured to be coupled to the intra-oral device to be at least partially exposed to an outer surface of the intra-oral device.

The present disclosure relates to an intra-oral device, wherein the sensor is configured to contact the soft tissue or the hard tissue in the oral cavity.

The present disclosure relates to an intra-oral device, wherein the sensor is a flexible sensor.

The present disclosure relates to an intra-oral device, wherein the sensor is not expandable or stretchable.

The present disclosure relates to an intra-oral device, wherein the intra-oral device includes a recess on a surface of the intra-oral device, and wherein the sensor is configured to be coupled to the recess.

The present disclosure relates to an intra-oral device, wherein the recess includes a slot, a groove, or an insert, and wherein the sensor is configured to be coupled to the slot, the groove, or the insert.

The present disclosure relates to an intra-oral device, wherein the intra-oral device includes a curved surface, wherein the sensor is an inflexible sensor, or a flat sensor, or a non-curved sensor, wherein the sensor is positioned in the intra-oral device with respect to the curved surface of the intra-oral device.

The present disclosure relates to an intra-oral device, wherein the curved surface of the intra-oral device corresponds to a curve of a dental arch in the oral cavity.

The present disclosure relates to an intra-oral device, wherein the device includes data storage operably linked to or connected to the sensor.

The present disclosure relates to an intra-oral device, wherein the data storage is built into a Radio Frequency Identity (RFID) chip or Near-Field Communication (NFC) chip.

The present disclosure relates to an intra-oral device, wherein the recess is from about 0.5 to about 15 mm deep.

The present disclosure relates to an intra-oral device, wherein a maximum distance between a surface of the sensor facing toward the soft tissue or the hard tissue and an outer surface of the intra-oral device is from about 0.5 mm to about 5 mm.

The present disclosure relates to an intra-oral device, wherein the sensor is a photoplethysmography (PPG) sensor, a physiological sensor, a physical sensor, a chemical sensor, a proximity sensor, a vibration sensor, or a positional sensor.

The present disclosure relates to an intra-oral device, wherein the sensor includes a silicone, plastic (optionally, an acetate), a liquid crystal polymer (LCP) substrate, an LCP base material adhered to a cladding (optionally a thin copper cladding), a fiber or a yarn, paper (optionally including cellulose nanofibers (CNFs), a polyaniline nanofiber/graphite nanofiber (PANI/GNF) nanocomposite, or a polyaniline (PANI) supportive matrix, or a combination thereof.

The present disclosure relates to an intra-oral device, wherein the intra-oral device is a mandibular advancement device (MAD), or an orthodontic device, or an intra-oral device that repositions a mandible or a maxilla.

In some embodiments, provided are products of manufacture, or intra-oral or dental devices, for example, orthodontic devices and mandibular advancement devices (MADs), having flexible sensors positioned on surface of the dental device such that the flexible sensor follows or is in close contact with the curved (or non-straight) contour of a patient's hard or soft tissues; and by being able to follow the contours of the patient's hard or soft tissue the flexible sensor is able to take more accurate readings of the patient's physiologic state.

For example, in some embodiments, photoplethysmography (PPG) sensors can be used in products of manufacture, or intra-oral or dental devices, for example, as provided herein to measure volumetric variations of blood circulation, and because PPG sensors use optical measurements using a light source and a photodetector at the surface of skin to measure and monitor volumetric variations of blood circulation and heart rate, having the PPG follow the contour of a patient's soft tissues results in relatively more accurate measurements.

In some embodiments, using a PPG mounted or placed on a flexible sensor allows measurement of physiologic data with greater precision.

In some embodiments, provided are products of manufacture manufactured as an intra-oral or dental device. In some embodiments, the product of manufacture may comprise a flexible sensor positioned on or adhered to or implanted in an outer surface of the intra-oral device configured such that the flexible sensor in is substantially complete, close contact with a patient's soft tissue or hard tissue (e.g., a tooth structure) when the intra-oral device is positioned or placed in the mouth or the oral cavity of the patient. In some embodiments, the sensor, while flexible, may not be expandable or stretchable (i.e., the sensor while flexible otherwise has a fixed dimension). In some embodiments, the product of manufacture may comprise and the flexible sensor is positioned in or substantially within a slot, groove or inset, on a surface of the intra-oral device. In some embodiments, the product of manufacture may comprise an inflexible, or flat, or non-curved sensor positioned on a curved surface of the intra-oral device. In some embodiments, the product of manufacture may comprise a sensor positioned in or substantially within a slot, groove or inset, on a surface of the intra-oral device, and optionally the curve of the surface of the intra-oral device substantially follows the curve of a dental arch.

In some embodiments, the product of manufacture may comprise a data storage capability or data storage unit operably linked to or connected to the flexible sensor, optionally a data storage capability built into a Radio Frequency Identity (RFID) chip or Near-Field Communication (NFC) chip.

In some embodiments, the slot, groove or inset is between about 0.5 to 15 mm deep, e.g., about 1 mm, about 2 mm, about 3 mm, about 4 mm, about 5 mm deep, about 6 mm, about 7 mm, about 8 mm, about 9 mm or about 10 mm deep. In some embodiments, in the products of manufacture, a maximum length from the sensor, such as a flexible sensor, or an inflexible, or flat, or non-curved sensor, to the outer surface of the product of manufacture, or soft or hard tissue of the patient, is less than about 1 mm, for example, between about 0.5 mm to about 5 mm, e.g., about 0.5 mm, about 1 mm, about 2 mm, about 3 mm, or about 4 mm, resulting in measurements by the sensor, such as a flexible sensor, or an inflexible, or flat, or non-curved sensor, of physiologic data with relatively greater precision than if the flexible sensor, or the inflexible, or flat, or non-curved sensor, were not in substantially close contact with the soft or hard tissue of the patient.

In some embodiments, the sensor, such as a flexible sensor, or an inflexible, or flat, or non-curved sensor may include a variety of types of sensors, with various functions and various measurement capabilities. For example, in some embodiments, the sensor may be a photoplethysmography (PPG) sensor, a physiological sensor, a physical sensor, a chemical sensor, a proximity sensor, vibration sensor, a positional sensor, or a combination thereof.

In some embodiments, the sensor, such as a flexible sensor, or an inflexible, or flat, or non-curved sensor may be manufactured using a silicone, plastic (optionally, an acetate), a liquid crystal polymer (LCP) substrate, an LCP base material adhered to a cladding (optionally a thin copper cladding), a fiber or a yam, paper (optionally comprising cellulose nanofibers (CNFs), a polyaniline nanofiber/graphite nanofiber (PANI/GNF) nanocomposite, a polyaniline (PANI) supportive matrix (optionally deposited on a core of optical fiber which acts as an active cladding), a vinylidene fluoride-trifluoroethylene-hexafluoroacetone copolymer, or a combination thereof.

In some embodiments, the product of manufacture or the intra-oral device may be a mandibular advancement device (MAD) or an orthodontic device, or an intra-oral device that repositions the mandible or the maxilla.

The present disclosure relates to an intra-oral device including: a sensor to be positioned to be adjacent to soft tissue or hard tissue in an oral cavity when the intra-oral device is positioned in the oral cavity.

The present disclosure relates to an intra-oral device, wherein the intra-oral device is to couple to the oral cavity of a patient to be positioned in the oral cavity.

The present disclosure relates to an intra-oral device, wherein the sensor is to obtain information from the adjacent soft tissue or the adjacent hard tissue when the intra-oral device is positioned in the oral cavity.

The present disclosure relates to an intra-oral device, further including a device to be operably linked to or connected to the sensor

The present disclosure relates to an intra-oral device, the device is operably linked to or connected to the sensor to receive the obtained information.

The present disclosure relates to an intra-oral device, wherein the device is disposed outside the oral cavity.

The present disclosure relates to an intra-oral device, wherein the sensor is to be coupled to the intra-oral device to be at least partially exposed to an outer surface of the intra-oral device.

The present disclosure relates to an intra-oral device, wherein the sensor is to contact the soft tissue or the hard tissue in the oral cavity.

The present disclosure relates to an intra-oral device, wherein the sensor is a flexible sensor.

The present disclosure relates to an intra-oral device, wherein the sensor is not expandable or stretchable

The present disclosure relates to an intra-oral device, wherein the intra-oral device include a recess on a surface of the intra-oral device, and wherein the sensor is to be coupled to the recess.

The present disclosure relates to an intra-oral device, wherein the recess includes a slot, a groove, or an insert, and wherein the sensor is to be coupled to the slot, the groove, or the insert.

The present disclosure relates to an intra-oral device, wherein the intra-oral device includes a curved surface, wherein the sensor is an inflexible sensor, or a flat sensor, or a non-curved sensor, wherein the sensor is positioned in the intra-oral device with respect to the curved surface of the intra-oral device.

The present disclosure relates to an intra-oral device, wherein the curved surface of the intra-oral device corresponds to a curve of a dental arch in the oral cavity.

The present disclosure relates to an intra-oral device, wherein the device includes data storage operably linked to or connected to the sensor.

The present disclosure relates to an intra-oral device, wherein the data storage is built into a Radio Frequency Identity (RFID) chip or Near-Field Communication (NFC) chip.

The present disclosure relates to an intra-oral device, wherein the recess is from about 0.5 to about 15 mm deep.

The present disclosure relates to an intra-oral device, wherein a maximum distance between a surface of the sensor facing toward the soft tissue or the hard tissue and an outer surface of the intra-oral device is from about 0.5 mm to about 5 mm.

The present disclosure relates to an intra-oral device, wherein the sensor is a photoplethysmography (PPG) sensor, a physiological sensor, a physical sensor, a chemical sensor, a proximity sensor, a vibration sensor, or a positional sensor.

The present disclosure relates to an intra-oral device, wherein the sensor includes a silicone, plastic (optionally, an acetate), a liquid crystal polymer (LCP) substrate, an LCP base material adhered to a cladding (optionally a thin copper cladding), a fiber or a yarn, paper (optionally including cellulose nanofibers (CNFs), a polyaniline nanofiber/graphite nanofiber (PANI/GNF) nanocomposite, or a polyaniline (PANI) supportive matrix, or a combination thereof.

The present disclosure relates to an intra-oral device, wherein the intra-oral device is a mandibular advancement device (MAD), or an orthodontic device, or an intra-oral device that repositions a mandible or a maxilla.

The present disclosure relates to an intra-oral device including: a device body configured to couple to dentition; and a sensor configured to couple to the device body to be positioned to be adjacent to soft tissue or hard tissue in an oral cavity when the device body couples to the dentition.

The present disclosure relates to an intra-oral device, wherein the sensor is configured to obtain information from the adjacent soft tissue or the adjacent hard tissue when the device body couples to the dentition.

The present disclosure relates to an intra-oral device, further including a device configured to be operably linked to or connected to the sensor to receive the obtained information.

The present disclosure relates to an intra-oral device, wherein the device is disposed outside the oral cavity.

The present disclosure relates to an intra-oral device, wherein the sensor is configured to be coupled to the intra-oral device to be at least partially exposed to an outer surface of the intra-oral device.

The present disclosure relates to an intra-oral device, wherein the sensor is configured to contact the soft tissue or the hard tissue in the oral cavity.

The present disclosure relates to an intra-oral device, wherein the sensor is a flexible sensor.

The present disclosure relates to an intra-oral device, wherein the sensor is not expandable or stretchable.

The present disclosure relates to an intra-oral device, wherein the intra-oral device includes a recess on a surface of the intra-oral device, and wherein the sensor is configured to be coupled to the recess.

The present disclosure relates to an intra-oral device, wherein the recess includes a slot, a groove, or an insert, and wherein the sensor is configured to be coupled to the slot, the groove, or the insert.

The present disclosure relates to an intra-oral device, wherein the intra-oral device includes a curved surface, wherein the sensor is an inflexible sensor, or a flat sensor, or a non-curved sensor, wherein the sensor is positioned in the intra-oral device with respect to the curved surface of the intra-oral device.

The present disclosure relates to an intra-oral device, wherein the curved surface of the intra-oral device corresponds to a curve of a dental arch in the oral cavity.

The present disclosure relates to an intra-oral device, wherein the device includes data storage operably linked to or connected to the sensor.

The present disclosure relates to an intra-oral device, wherein the data storage is built into a Radio Frequency Identity (RFID) chip or Near-Field Communication (NFC) chip.

The present disclosure relates to an intra-oral device, wherein the recess is from about 0.5 to about 15 mm deep.

The present disclosure relates to an intra-oral device, wherein a maximum distance between a surface of the sensor facing toward the soft tissue or the hard tissue and an outer surface of the intra-oral device is from about 0.5 mm to about 5 mm.

The present disclosure relates to an intra-oral device, wherein the sensor is a photoplethysmography (PPG) sensor, a physiological sensor, a physical sensor, a chemical sensor, a proximity sensor, a vibration sensor, or a positional sensor.

The present disclosure relates to an intra-oral device, wherein the sensor includes a silicone, plastic (optionally, an acetate), a liquid crystal polymer (LCP) substrate, an LCP base material adhered to a cladding (optionally a thin copper cladding), a fiber or a yarn, paper (optionally including cellulose nanofibers (CNFs), a polyaniline nanofiber/graphite nanofiber (PANI/GNF) nanocomposite, or a polyaniline (PANI) supportive matrix, or a combination thereof.

The present disclosure relates to an intra-oral device, wherein the intra-oral device is a mandibular advancement device (MAD), or an orthodontic device, or an intra-oral device that repositions a mandible or a maxilla.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

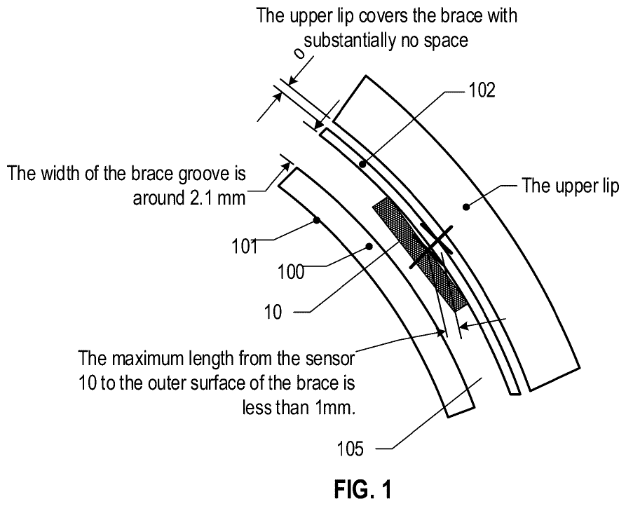

FIG. 1 illustrates a Photoplethysmography (PPG) sensor as a sensor 10 that is mounted in or on a flexible circuit board for a mandibular advancement device as an intra-oral device 100 in some embodiments. In some embodiments, products of manufacture, or intra-oral or dental devices, as provided herein, are configured as illustrated in FIG. 1, where a PPG sensor is mounted in or on a flexible circuit board. Referring to FIG. 1, the products of manufacture, or intra-oral or dental devices, as an intra-oral device 100, can be labeled as a brace. Both the outer surface and the inner surface of the product of manufacture, or brace, can be curved or contoured to substantially follow the curvature or variations of the patient's soft tissue. In some embodiments, the soft tissue may include various types of soft tissue inside of the mouth or the oral cavity, including any soft tissue adjacent or between the teeth, including but not limited to the papilla, tissue of the upper and lower dental arches, marginal gingiva, gingival sulcus, inter-dental gingiva, gingival gum structure on lingual and buccal surfaces up to and including the muco-gingival junction and/or the palate and/or the floor of the mouth. In some embodiments, the soft tissue area may include the muco-buccal folds, hard and soft palates, lining mucosa, the tongue and/or attached gingival tissue. In some embodiments, the inner surface 101 of the brace as the intra-oral device 100 may be pressed close to the tooth. In some embodiments, the other surface 102 of the brace as the intra-oral device 100 can face toward the upper lip. In some embodiments, the upper lip covers the outer surface 102 of the intra-oral device 100, substantially providing no space in between the upper lip and the outer surface 102 of the intra-oral device 100. In some embodiments, the maximum length from the sensor 10 to the outer surface 102 of the intra-oral device 100 as the brace can be less than 1 mm. In some embodiments, a groove as a recess 105 is comprised in the intra-oral device 100. In some embodiments, the width of the groove 105 can be about 2 mm, e.g., 2.1 mm.

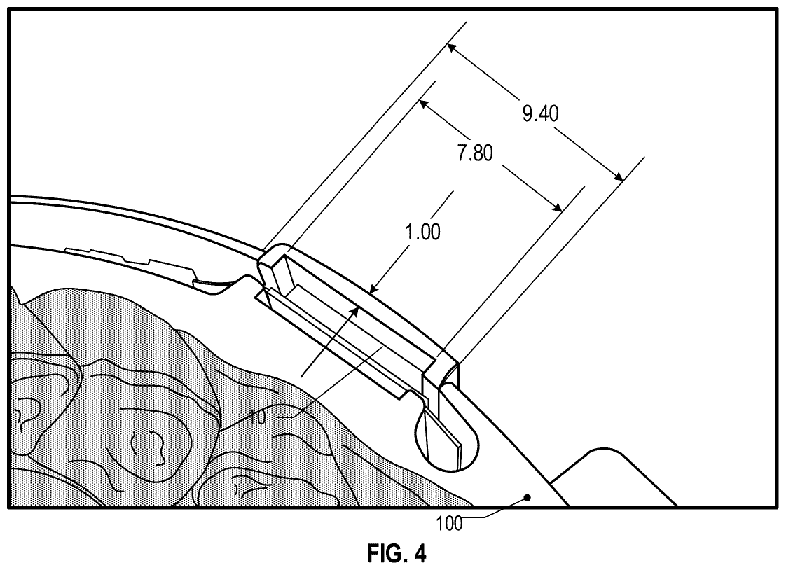

In some embodiments, the flexible circuit board with the sensor or sensors (such as a PPG or any other sensor) can be placed in a groove or inset cut or otherwise designed into either/or the outer surface of the product of manufacture. In some embodiments, the width of the groove or insert can be from about 0.5 mm to about 10 mm, such as about 2.1 mm, about 3 mm, etc. In some embodiments the depth of the inset, slot or groove is between about 0.5 to 15 mm deep, or is about 1 mm, 2 mm, 3 mm, 4 mm, 5 mm deep, 6 mm, 7 mm, 8 mm, 9 mm or 10 mm deep. For example, FIG. 4 illustrates a flexible circuit board with the sensor 10 or sensors 10 placed in a groove 105 or inset cut 105 or slot 105, or otherwise designed into either/or the outer surface of an intra-oral device 100 as a product of manufacture in some embodiments. An exemplary product of manufacture having an inset, slot or groove is illustrated in FIG. 4.

In some embodiments, the patient's soft tissue, for example, the mucosa of the inside of the lips, or soft tissue of the cheek, rest against the outer surface of the product of manufacture. In some embodiments, the product of manufacture can be designed such that there is no space, or minimal space, or substantially no space (for example, between about 0.5 mm and about 5 mm), between the outer surface of the product of manufacture and the patient's soft tissue. In some embodiments, this is accomplished by designing the outer surface of the product of manufacture to have a curvature that is substantially the same as the curvature of the patient's soft tissue. In some embodiments, because of the curved design of the product of manufacture and the flexible sensor, the maximum length from the sensor (a PPG in this example) to the outer surface of the product of manufacture (or brace) is less than about 1 mm (or is between about 0.25 and 1.5 mm), resulting in measurements of physiologic data with greater precision.

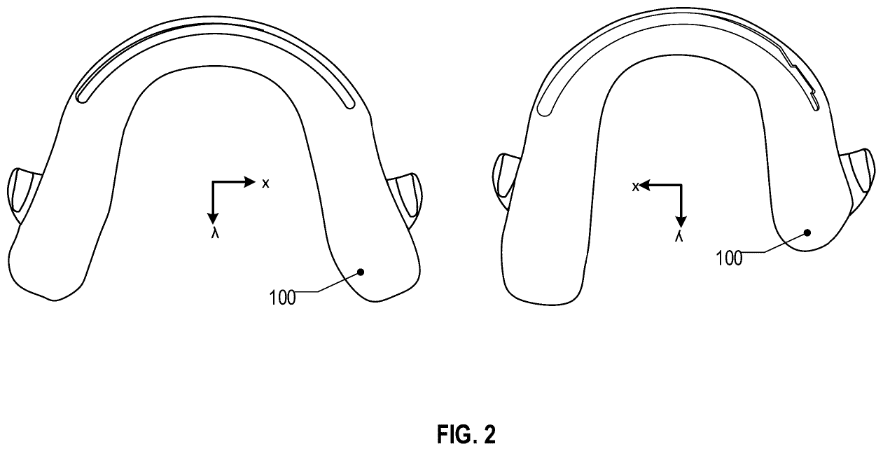

FIG. 2 illustrates a flexible circuit board configurations for two different arch forms with changed radiuses of the slot cut to allow insertion of a flexible sensor circuit board 10 in some embodiments. In some embodiments, the flexible circuit board configuration 10 is also designed and manufactured to follow the patient's dental arch (mandibular and/or maxillary), for example, by varying radius of circuit board slot cut, for example, as illustrated in FIG. 2, which illustrates two very different arch forms; however, the radius of the slot cut was changed in each to allow insertion of a flexible sensor circuit board (for example, a PPG sensor circuit board).

In some embodiments, the sensor is an optical device or member (for example, can be a light source or light meter or light detecting element, such as for example a LED light source or LED detecting element).

In some embodiments, the sensor or the flexible circuit board configuration may not follow the curvature of the product of manufacture (for example, the exemplary intra-oral dental device does not follow the arch curvature, i.e., the sensor or the flexible circuit board is flat as compared to the intra-oral dental device that substantially follows the curve of the dental arch), for example, as illustrated by the exemplary intra-oral device 100 as a product of manufacture shown in FIG. 4, which shows a flat LED light source. In some embodiments, by not following the curvature of the product of manufacture, for example, not following the arch curvature, the sensor (for example, a PPG sensor area), or a LED light source (for example, the flat or non-curved sensor, light source, light meter), has a stronger signal (as compared to a curved or non-flat equivalent); and this configuration may deter light and/or signal from spreading out and helps returned light signal to be stronger; this will increase device signal precision.

In some embodiments, the flexible sensor can be between about 1 and 40 mm or 2 and 20 mm in length, and the flexible sensor is in close contact with, or actually contacts, a patient's hard or soft tissue substantially most of the length of the flexible sensor. In some embodiments, the flexible sensor is between about 1 and 10 mm or 2 and 20 mm in width, and the flexible sensor is in close contact with, or actually contacts, a patient's hard or soft tissue substantially most of the length and width of the flexible sensor.

In some embodiments, the product of manufacture, or intra-oral or dental device, as provided herein, can have data storage capability (or data storage unit). In some embodiments, the sensor (for example, a PPG sensor) can be operatively linked or connected to data storage capability (or a data storage unit), and the sensor sends measurements to the data storage unit, which retains this data, and optionally the data storage unit transfers the data in real time, or at a later designated time, for example, in the morning when the data is accrued during the night to a remote source, for example, by wireless communication, for example, using near-field communication (NFC) and/or Bluetooth (BLE) technologies, including for example, a NFC communication module and an NFC antenna. In some embodiments, this exemplary data transfer method can allow a minimized battery size, one benefit of which is improved patient comfort by having a smaller sized battery and thus a smaller sized intra-oral product of manufacture. The wireless communication can be in the form of a radio, an infrared, or a magnetic communication.

In some embodiments, monitoring of data from the sensors can be continuous (or always on) or can be sampled at a regular frequency (for example, between 0.001 Hz and 1 KHz, between 1 and 120 times/hour, between 1 and 24 time/day, etc.) or can be sampled for a discrete time after inserting the apparatus. In some embodiments, monitoring may comprise monitoring over a time period of greater than one day (for example, more than: 24 hours, 36 hours, 48 hours, 3 days, 4 days, 5 days, etc.). In some embodiments, monitoring may be continuous (for example, at periodic intervals, e.g. 100 Hz, 10 Hz, 1 Hz, 1/min, every 2 min, every 3 min, every 5 min, every 10 min, every 30 min, every hour, etc.) or at discrete intervals (for example, when requested by a user, etc.).

In some embodiments, the wireless communication can be in various forms, such as in the form of a radio, an infrared, a magnetic communication and/or near field communication (NFC), including NFC-to-NFC communication, for example, Wi-Fi, radio (RF, UHF, etc.), infrared (IR), microwave, Bluetooth (including Bluetooth low energy or BLE), magnetic field induction (including for example NFC), Worldwide Interoperability for Microwave Access (WiMAX), zigbee (a standards-based wireless technology that enables low-cost, low-power wireless machine-to-machine (M2M) and internet of things (IoT) networks) and/or ultrasound.

In some embodiments, systems as provided herein may comprise a monitoring apparatus with a first antenna within a housing of a near field communication (NFC) to Bluetooth communication (e.g., BLE) signal coupler device, transmitting the monitoring data from the monitoring apparatus to the NFC to BLE signal coupler device by NFC, and retransmitting the monitoring data from the NFC to BLE signal coupler device via a Bluetooth signal to a handheld electronic device. In some embodiments, systems as provided herein may comprise inserting the monitoring apparatus into the NFC to BLE signal coupler device, wherein the NFC to BLE signal coupler device is configured as a case configured to hold the monitoring apparatus (or MA and a dental appliance to which the monitoring apparatus is coupled). In some embodiments, systems as provided herein may comprise receiving the Bluetooth signal in the handheld electronic device, wherein the handheld electronic device comprises a smartphone. In some embodiments, systems as provided herein may comprise modifying the monitoring data before retransmitting the data, and transmitting the monitoring data may comprise receiving the NFC signal comprising the monitoring data on a first antenna of the NFC to BLE signal coupler device. In some embodiments, retransmitting the monitoring data may comprise transmitting the monitoring data as the Bluetooth data via a second antenna of the NFC to BLE signal coupler device configured for Bluetooth communication.

In some embodiments, the sensor and the data storage capability are operatively linked or connected to a power source, such as a battery, such as a lithium-ion battery or a lithium-thionyl chloride battery. In some embodiments, the battery is a rechargeable or replaceable battery, and can also comprise a battery recharging circuit compatible with industry standards.

In some embodiments, product of manufacture, or intra-oral or dental device, as provided herein, comprise in addition to a power source: an on-board memory, a communication module, an analog/digital converter, a control module for activating stepper motors, and/or an I/O bus to connect to external components.

In some embodiments, the data storage capability (or data storage unit), for example, a data storage capability built into a Radio Frequency Identity (RFID) chip or Near-Field Communication (NFC) chip, comprises for memory an Electronic Product Code (EPC) memory, which is a writeable memory to store an EPC code having a minimum potential of 96 bits. In some embodiments, the data storage capability is a reserved memory designed to lock the reading and writing activities of a radio frequency identity (RFID) chip, for example, an ultra-high-frequency (UHF) RFID. In some embodiments, the data storage capability can also comprise tag identifier (TID) memory, a read-only memory (ROM), to differentiate one tag from another. In some embodiments, the data storage capability can comprise a second writable memory bank that allows users to read already entered data, and write more data through an RFID or NFC reader.

In some embodiments, the product of manufacture, or intra-oral or dental device, as provided herein, may comprise a rechargeable or removable power source and one or more coupling coils arranged in a power/communication circuit such that the power source can provide electrical power for exciting coupling coils. Exciting coils can create a magnetic field that can reach one or more sensors; and, a coil and/or an antenna can be excited by the electromagnetic field generated by coupling coils, which induces a current in the coil/antenna. Through inductive coupling, the induced current can be used to power the sensor for an extended period of time. For example, a rechargeable battery can provide as much as eight hours of power to a sensor through this inductive coupling configuration.

In some embodiments, the product of manufacture, or intra-oral or dental device, as provided herein, comprises an external recharging device that incorporates wireless charging functionality by way of a wireless charging/coupling pad or coil incorporated into housing of an external recharging cradle to re-charge the product of manufacture, or intra-oral or dental device. Current from an internal or external power source can flow through the coil/pad inside recharging device creating an electromagnetic field, and the electromagnetic field can induce current in a coupling coil (for example, an inductive coupling) of the product of manufacture, or intra-oral or dental device, that is in electrical communication with the battery. Such current can recharge the battery of the intra-oral appliance. As such, the external recharging device can provide wireless recharging of the remote controller of the product of manufacture, or intra-oral or dental device.

In some embodiments, the external recharging device can also provide gentle ultrasonic or chemical cleaning of the teeth covering of the product of manufacture, or intra-oral or dental device. The cleaning can be similar to other ultrasonic or chemical-based cleaning system. The external recharging device can include a display that indicates that the charging and cleaning function is either “off,” “in progress” or “complete” by way of a multi-color indicator, for example. The wireless recharging function can provide a complete charge in 120 minutes using a 5V, 2A power source, for example.

In some embodiments, a miniaturized radio transponder and radio receiver and transmitter assembly, such as a radio frequency identity (RFID or NFC) chip, operatively connected or linked to a miniature antenna to wirelessly transmit, and optionally also receive, data (such as instructions) to and from a remote device (or base) such as a computer (such as a laptop computer or a desktop computer), a smart tablet, or a phone, such as a smart phone, which can have software, optionally software on an app; where optionally the data is read and instructions are given back to the product of manufacture by a medical professional, for example, a dentist.

In some embodiments, the base analyzes the wirelessly received data and, based on a pre-scripted routine or program, relays a command to the product of manufacture, or intra-oral device, such as a MAD, to take an action in response. In some embodiments, wherein the product of manufacture, or intra-oral device, such as a MAD, further comprises a motor, and the action comprises a communication to the motor from the base to activate a change in the positional relationship of an upper (maxillary) splint positioned on the upper dentition to the lower (mandibular) splint positioned on the lower dentition. In some embodiments, the at least one sensor comprises a communication component configured for wireless communication with a base, and the base and the mandibular advancement device operate in a feedback system, and when data obtained by the at least one sensor is communicated with the base, the base analyzes the data and, based on a pre-scripted routine, relays a command to the mandibular advancement device to take an action in response, and the action comprises a communication to the motor from the base to activate a change in the positional relationship of the upper splint positioned on the upper dentition to the lower splint positioned on the lower dentition.

In some embodiments, the product of manufacture, or intra-oral device, such as a MAD, as provided herein, comprises elements to record data, and transmit recorded data to storage for review and analysis, where the transferred data may be subjected to downloading, validation, storage, analysis, measuring, database fusion, data output, and data recording. Output may be further transferred to a computer, processor, phone, tablet computer, or other hardware for review by a user or technical, research, medical, or other oversight personnel; data also can be transmitted to the product of manufacture, including firmware or other onboard operating instructions.

In some embodiments, provided are systems (or multiplex systems) comprising a product of manufacture, or intra-oral device, such as a MAD, as provided herein, and one or more computers or devices loaded with a set of the computer-executable instructions. The computers or devices may be general purpose computers, or special-purpose computers, or other programmable data processing apparatus to produce a particular machine, such that the one or more computers or devices are instructed and configured to carry out required calculations, processes, steps, operations, algorithms, statistical methods, formulas, or computational routines to practice methods as provided herein. The computer or device performing the specified calculations, processes, steps, operations, algorithms, statistical methods, formulas, or computational routines may comprise at least one processing element such as a central processing unit (or processor) and a form of computer-readable memory which may include random-access memory (RAM) or read-only memory (ROM). The computer-executable instructions can be embedded in computer hardware or stored in the computer-readable memory such that the computer or device may be directed to perform one or more of the calculations, steps, processes and operations.

In some embodiments, the system as provided herein that comprises a computer (for example, a desktop computer, a portable computer, such as a tablet, laptop, PDA, or smartphone, or a set of computers) is also operably connected to a network including a client-server configuration and one or more database servers. The network may use any suitable network protocol, including IP, UDP, or ICMP, and may be any suitable wired or wireless network including any local area network, wide area network, Internet network, telecommunications network, Wi-Fi enabled network, or Bluetooth enabled network. In one embodiment, the computer system comprises a central computer connected to the internet that has the computer-executable instructions stored in memory that is operably connected to an internal electronic database. The central computer may perform the computer-implemented method based on input and commands received from remote computers through the internet. The central computer may effectively serve as a server and the remote computers may serve as client computers such that the server-client relationship is established, and the client computers issue queries or receive output from the server over a network.

In some embodiments, a system as provided herein also comprises a graphical user interface (GUI) which may be used in conjunction with a computer-executable code and electronic databases. The graphical user interface may allow a user to perform these tasks through the use of text fields, check boxes, pull-downs, command buttons, and the like; and graphical features may be implemented for performing methods as provided herein. The user interface may optionally be accessible through a computer connected to the internet. In one embodiment, the user interface is accessible by typing in an internet address through an industry standard web browser and logging into a web page. The user interface may then be operated through a remote computer (for example, a computer operated by a dentist) accessing the web page and transmitting queries or receiving output from a server through a network connection.

In some embodiments, the product of manufacture, or intra-oral device, such as a MAD, as provided herein, comprises one or more flexible sensors, wherein the sensor can be a photoplethysmography (PPG) sensor, a physiological sensor, a physical sensor, a chemical sensor, a proximity sensor, vibration sensor, or a positional sensor.

In some embodiments, the physiological sensor detects and measures patient physiological parameters (data) comprising for example: blood sugar, blood oxygen levels, body temperature, respiration rate and/or heart rate.

In some embodiments, the physical sensor is able to detect and measure: vibration in the breathing, airflow rate, oxygen concentration of inhaled air, carbon dioxide concentration of exhaled air, atmospheric pressure, air pressure inside the patient's oral cavity, noise, pressure exerted on the MAD by the patient's teeth, and/or actigraphic data (actigraphic measurements comprise sleep parameters and average motor activity using a noninvasive accelerometer).

In some embodiments, the chemical sensor detects and measures: saliva pH, saliva glucose concentration, saliva conductivity, stress markers, salivary cortisol, blood oxygen saturation level, blood pH, blood glucose levels, blood insulin levels, and/or inflammatory markers.

In some embodiments, the positional sensor detects and records a position of the product of manufacture, or intra-oral device, such as a MAD, in a mouth with respect to a predetermined reference location.

In some embodiments, a proximity sensor can comprise one or more of a capacitive sensor, an eddy-current sensor, a magnetic sensor, an optical sensor, a photoelectric sensor, an ultrasonic sensor, a Hall Effect sensor, an infrared touch sensor, or a surface acoustic wave (SAW) touch sensor, and the one or more proximity sensors may be configured to generate sensing data when in proximity to one or more of the patient's enamel, gingiva, oral mucosa, cheeks, lips, or tongue; and the one or more proximity sensors may be integrated with an intra-oral device or appliance as provided herein, coupled to a soft tissue or a tooth, or a combination thereof. In some embodiments,

In some embodiments, a vibration sensor comprises one or more of: a MEMS microphone, an accelerometer, or a piezoelectric sensor; and intra-oral vibration patterns may be associated with one or more of: vibrations transferred to the patient's teeth via the patient's jaw bone, teeth grinding, speech, mastication, breathing, or snoring; and the processor may determine whether the intra-oral appliance is being worn by comparing the intra-oral vibration patterns to patient-specific intra-oral vibration patterns; and the one or more vibration sensors may be integrated with the intra-oral appliance, coupled to a tooth, or a combination thereof; and the processor can be integrated with the intra-oral device or appliance as provided herein or coupled to soft tissue or a tooth.

In some embodiments, the flexible sensor comprises: a touch or tactile sensor such as a capacitive or resistive sensor, a proximity sensor, an audio sensor (for example, a microelectromechanical system (MEMS) microphone), a color sensor (for example, a RGB color sensor), an electromagnetic sensor (for example, a magnetic reed sensor, a magnetometer), a light sensor, a force sensor (for example, a force-dependent resistive materials), a pressure sensor, a temperature sensor, a motion sensor (for example, an accelerometer and/or a gyroscope), a vibration sensor, a piezoelectric sensor, a strain gauge, a pH sensor, a conductivity sensor, a gas flow sensor, a gas detection sensor, a humidity or moisture sensor, a physiological sensor (for example, an electrocardiogramsor, a bio-impedance sensor, a photo-plethysmography sensor, a galvanic skin response sensor), or combinations thereof.

In some embodiments, motion sensors comprise or are designed as accelerometers, gyroscopes, piezoelectric film vibration sensors, gravity sensors, and microwave emitters and/or receivers. The motion sensors can be integrated into a product of manufacture (such as an intra-oral appliance) as provided herein worn on a patient's upper or lower jaw, or can be distributed across an appliance worn on the upper jaw and an appliance worn on the lower jaw. In some embodiments, the motion sensors are configured to generate data representative of the patient's jaw movement patterns, and the monitoring device processes and analyzes the movement patterns (for example, using power spectrum and/or kinematic analysis) to determine whether the patterns indicate that the appliance(s) are being worn. In some embodiments, the monitoring sensor can distinguish jaw movement patterns associated with different oral activities (for example, mastication, grinding, speech, etc.).

In some embodiments, flexible sensors as used in products of manufacture as provided herein are operatively connected to or configured as a switch that is activated and/or deactivated in response to a particular type of signal or stimulus such as an optical, electrical, magnetic and/or mechanical signal or stimulus.

In some embodiments, the flexible sensor is manufactured using a silicone, plastic (for example, an acetate) or liquid crystal polymer (LCP) substrate (see for example, Wang et al Polymers (Basel) (2018) July; 10(7): 694) base material adhered to a cladding such as a thin copper cladding, a fiber or a yam (see for example, Li et al nano micro Small vol. 18 (7) (2022)), paper (such as cellulose nanofibers (CNFs) or paper, see for example, Liu et al, J. Materials Chem. 10(10)), a polyaniline nanofiber/graphite nanofiber (PANI/GNF) nanocomposite (for example, see Mohammed et al IEEE Access, vol. 9, pg 145282 (2021), or a polyaniline (PANI) supportive matrix which can be deposited on a core of optical fiber which acts as an active cladding (see for example, Pahurkar et al, Measurement, Vol. 61, February 2015, Pg 9-15). In some embodiments, the flexible sensor is manufactured using a cladding material comprising a vinylidene fluoride-trifluoroethylene-hexafluoroacetone copolymer (see for example, EP88308227B1). The disclosures of the foregoing references are incorporated herein by reference in their entireties.

In some embodiments, the flexible sensor is manufactured using (or comprises) an electroconductive medical polymer such as: acrylic, acrylonitrile butadiene styrene (ABS), polycarbonate/acrylonitrile-butadiene-styrene terpolymer blend (PC/ABS), acetyl, polycarbonate, polyolefins such as polypropylene (PP) and polyethylene (PE) and other resin systems, polymers rendered electrically conductive, such as acetals such as polyoxymethylene (POM), acrylics such as poly(methyl methacrylate) (PMMA), fluoropolymers such as polytetrafluoroethylene (PTFE), polyvinylidene difluoride (PVDF) and perfluoroalkoxy alkanes (PFA), polycarbonate (PC) and PC alloys, polyethereketone (PEEK), polyolefins (PP), (PE), and polymethylpentene (PMP), polysulfone (PSU), polyethersulfone (PES), thermoplastic polyurethane elastomer (TPUR), and styrenics polystyrene (PS) and ABS. In some embodiments, the polymer comprises polypyrrole (PPy), polyaniline (PANI), poly(3,4-ethylenedioxythiophene) (PEDT, PEDOT), polythiophene (PTh), polythiophene-vinylene (PTh-V), poly(2,5-thienylenevinylene) (PTV), poly(3-alkylthiophene) (PAT), poly(p-phenylene) (PPP), poly-p-phenylene-sulphide (PPS), poly(p-phenylenevinylene) (PPV), poly(p-phenylene-terephthalamide) (PPTA), polyacetylene (PAc), poly(isothianaphthene) (PITN), poly(a-naphthylamine) (PNA), polyazulene (PAZ), polyfuran (PFu), polyisoprene (PIP), polybutadiene (PBD), and/or poly(3-octylthiophnene-3-methylthiophene) (POTMT); or, a PERMASTAT PLUS™ polymer, or as described in U.S. Pat. No. 8,685,461.

In some embodiments, the product of manufacture comprising the flexible sensor as provided herein is any intra-oral device, such as an orthodontic device, or a MAD, and in some embodiments the MAD is manufactured, designed and/or used as has been described in for example: U.S. Pat. Nos. (USPN) 11,617,677; 11,291,580; 11,207,207.

In some embodiments, the product of manufacture is made using three-dimensional (3D) printing and/or a computer aided design (CAD) computer program, for example comprising a method comprising: a) importing into a computer aided design (CAD) computer program a digitized data set obtained from a three-dimensional scan of a patient's dentition; b) preparing a three-dimensional electronic model of the patient's dentition; c) subtracting the three-dimensional electronic model of the patient's dentition from an image of a solid block to obtain an appliance data set; and d) manufacturing a dental appliance in accordance with the appliance data set. In some embodiments, the volume of the solid block correlates to the volume of the sensor or shaped to be sufficient to encompass the sensor.

In some embodiments, the product of manufacture is made using three-dimensional (3D) printing and/or a computer aided design (CAD) computer program, for example comprising a method comprising: a) preparing a three-dimensional electronic model of the flexible printed circuit board; b) importing into a computer aided design (CAD) computer program the three-dimensional electronic model of the flexible printed circuit board; c) subtracting the three-dimensional electronic model of the flexible printed circuit board from the appliance data set to create the custom slot in the device for the physical flexible circuit board, creating a male/female mirroring between the device slot and the physical flexible circuit board to facilitate proper sensor positioning and manufacturing assembly.

In some embodiments, the product of manufacture is manufactured from a polymer, a composite, a thermoplastic, a thermoset, and the like. In some embodiments, the appliance is subtractively manufactured from a block of, or additively manufactured to form, a material selected from the group consisting of standard polymethylmethacrylate (PMMA), lined PMMA, high-strength polyetheretherketone (PEEK), polymer produced from polyoxymethylene and acetal copolymers (DURACETAL®), glycol modified polyethylene terephthalate (PETg), and a physiologically compatible, water insoluble, non-malleable polymer, wood, and metal.

In some embodiments, the product of manufacture is manufactured in whole or in part from a polymer, a composite, a thermoplastic, a thermoset, and the like. In some embodiments, the appliance is subtractively manufactured from a block of, or additively manufactured to form, a material comprising: poly(butylene succinate) and copolymers thereof; polymethyl-methacrylate (PMMA); lined PMMA; high-strength polyether ether ketone (PEEK); a (meth)acryloyloxy-substituted benzoic acid ester; urethane di(meth)acrylate (UDMA); triethyleneglycol dimethacrylate (TEGDMA); tetraethylene glycol diacrylate (E4-A); trimethylolpropanetriacrylate (TTA); a polymer matrix comprising diurethanedimethacrylate (DUDMA) and glycerol dimethacrylate (GDMA); a polymer produced from polyoxymethylene and acetal copolymers (DURACETAL®); glycol modified polyethylene terephthalate (PETg); a physiologically compatible, water insoluble, non-malleable polymer; wood; plastic, and/or, a metal.

In some embodiments, the product of manufacture is manufactured suing an extrusion, and can be designed in CAD, or is designed and then merged together in CAD to make a desired and complete product of manufacture (for example, a MAD or a splint). In some embodiments, the designed feature of the product of manufacture comprises a standard and well-defined geometrical shape, for example a cube, a pyramid, a cone, a cylinder. A well-defined geometrical shape is one in which the cross section of a feature of the product of manufacture is a standard geometrical shape of a circle, a square, a rectangle, a parallelogram, a circle, a triangle, a rhombus, and the like. In other embodiments, a feature of the product of manufacture comprises a customized shape. The customized shape is one in which the cross section is a non-standard, or amorphous, geometrical shape. In some embodiments, a feature is entirely made up of one, or a merger of two or more, standard geometrical shapes. In other embodiments, a feature of the product of manufacture is entirely made up of one, or a merger of two or more, non-standard geometrical shapes. In other embodiments, the feature comprises between 10%, 20%, 30%, 40%, 50%, 60%, 70%, 80% or 90% of one or more standard geometrical shape(s), with the remainder being comprised of one or more non-standard shape(s). In other embodiments, a feature of the product of manufacture (for example, a tapered offset) is adjusted in an iterative process of addition and subtraction (for example at least two cycles of addition and subtraction) until an optimized design (e.g., a taper that fits the patient's dentition very well) is obtained. The artisan of ordinary skill understands that while the above processes are given an example for illustration, the design processes can be used with any feature of the product of manufacture or their accessories.

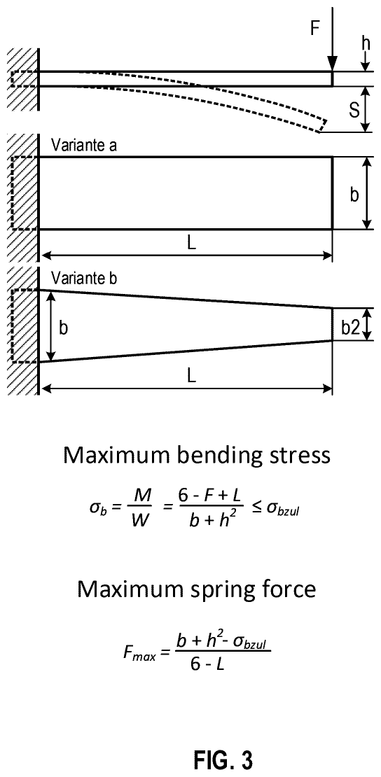

FIG. 3 illustrates example structures with added extra thickness to create more spring force to teeth which need torquing and rotating in some embodiments. In some embodiments, the product of manufacture as provided herein is milled as a clear aligner; and can also add extra thickness to create more spring force to teeth which need torquing and rotating, for example, as illustrated in FIG. 3.

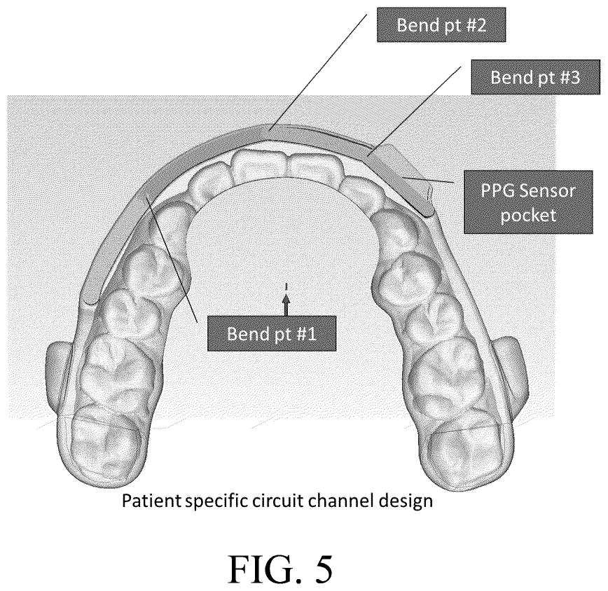

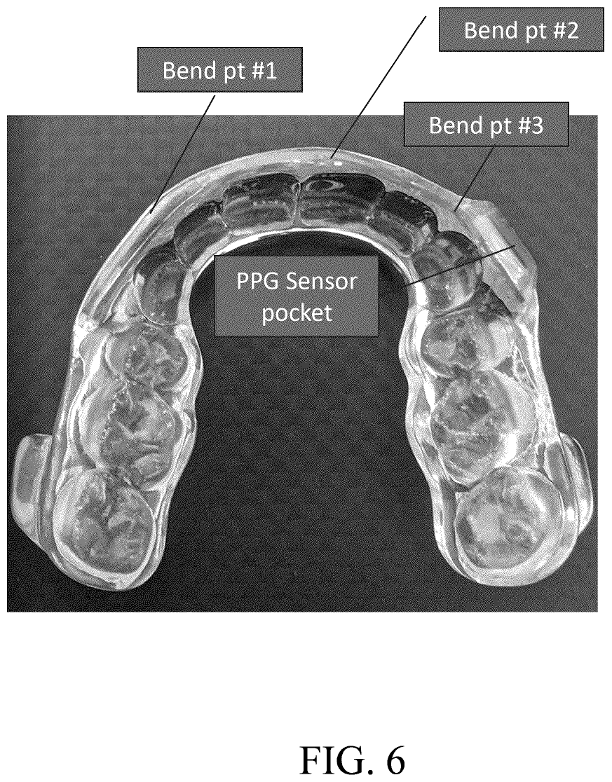

FIG. 5 illustrates an orthodontic device with a sensor pocket and a circuit channel design in some embodiments. Referring to FIG. 5, the orthodontic device can include a patient specific circuit design, for example, based on a relative position of a recess or a protrusion such as a sensor pocket and different bend points. FIG. 6 illustrates an orthodontic device with a sensor pocket and bend points in some embodiments. Referring to FIG. 6, the orthodontic device can include a circuit design, for example, based on a relative position of a recess or a protrusion such as a sensor pocket and different bend points.

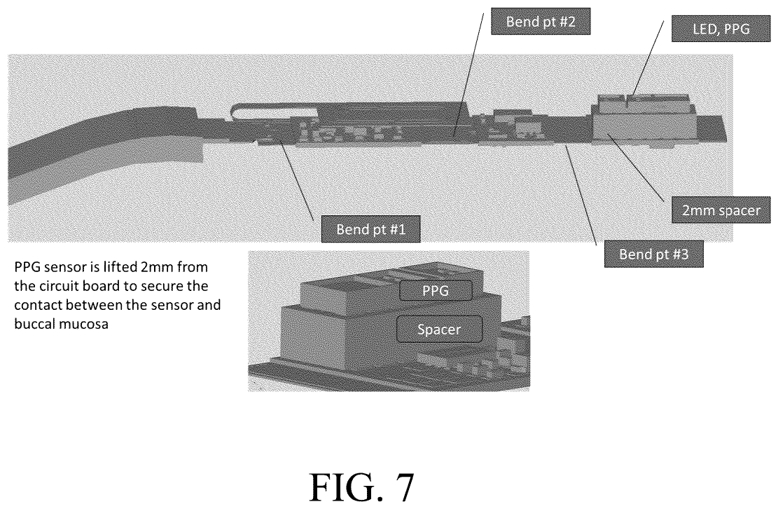

FIG. 7 illustrates a sensor design with bend points orthodontic device in some embodiments. Referring to FIG. 7, the sensor can be a variery of different types, such as a PPG sensor. For example, in some embodiments, the sensor can be provided with an LED light source and a spacer and can include different bend points. The sensor can be lifted about 2 mm from the circuit board to secure the contact between the sensor and buccal mucosa or to position the sensor to be close to, or to be adjacent to, or to be positioned with respect to buccal mucosa.

### Kits

Provided are kits comprising products of manufacture and systems (for example, multiplexed systems) for practicing methods as provided herein; and optionally, and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition; or in some embodiment, when the curve of the surface of the intra-oral device substantially follows the curve of a dental arch, the curve of the surface of the intra-oral device can have one or some sections of the device that do not exactly or closely follow the curve of a dental arch but 75%, 80%, 85%, 90%, 91%, 92%, 93%, 94%, 95%, 96%, 97%, 98%, 99% or 99.5% of the intra-oral device does follow the curve of a dental arch.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications.

Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

A number of embodiments of the invention have been described. Nevertheless, it can be understood that various modifications may be made without departing from the spirit and scope of the invention. Accordingly, other embodiments are within the scope of the following claims.

What is claimed is:

1. A device comprising:
a device body configured to couple to dentition; and
a sensor configured to couple to the device body to be positioned to be adjacent to soft tissue or hard tissue in an oral cavity when the device body couples to the dentition.

2. The device of claim 1, wherein the sensor is configured to obtain information from the adjacent soft tissue or the adjacent hard tissue when the device body couples to the dentition.

3. The device of claim 2, further comprising a device configured to be operably linked to or connected to the sensor to receive the obtained information.

4. The device of claim 3, wherein the device is disposed outside the oral cavity.

5. The device of claim 1, wherein the sensor is configured to be coupled to the device to be at least partially exposed to an outer surface of the device.

6. The device of claim 1, wherein the sensor is configured to contact or be positioned to be close to the soft tissue or the hard tissue in the oral cavity.

7. The device of claim 1, wherein the sensor is a flexible sensor.

8. The device of claim 7, wherein the sensor is not expandable or stretchable.

9. The device of claim 1, wherein the device includes a recess or a protrusion on a surface of the device, and wherein the sensor is configured to be coupled to the recess or the protrusion.

10. The device of claim 9, wherein the recess include the protrusion to push the sensor against the soft tissue or the hard tissue.

11. The device of claim 9, wherein the recess or the protrusion includes a slot, a groove, or an insert, and wherein the sensor is configured to be coupled to the slot, the groove, or the insert.

12. The device of claim 1, wherein the device includes a curved surface, wherein the sensor is an inflexible sensor, or a flat sensor, or a non-curved sensor, wherein the sensor is positioned in the device with respect to the curved surface of the device.

13. The device of claim 12, wherein the curved surface of the device corresponds to a curve of a dental arch in the oral cavity.

14. The device of claim 1, wherein the device includes data storage operably linked to or connected to the sensor.

15. The device of claim 14, wherein the data storage is built into a Radio Frequency Identity (RFID) chip or Near-Field Communication (NFC) chip.

16. The device of claim 9, wherein the recess or the protrusion has a buccal lingual thickness from about 0.5 to about 15 mm deep.

17. The device of claim 1, wherein a maximum distance between a surface of the sensor facing toward the soft tissue or the hard tissue and an outer surface of the device is from about 0.5 mm to about 5 mm.

18. The device of claim 1, wherein the sensor is a photoplethysmography (PPG) sensor, a physiological sensor, a physical sensor, a chemical sensor, a proximity sensor, a vibration sensor, or a positional sensor.

19. The device of claim 1, wherein the sensor includes a silicone, plastic, a liquid crystal polymer (LCP) substrate, an LCP base material adhered to a cladding, a fiber or a yarn, paper (optionally comprising cellulose nanofibers (CNFs), a polyaniline nanofiber/graphite nanofiber (PANI/GNF) nanocomposite, or a polyaniline (PANI) supportive matrix, or a combination thereof.

20. The device of claim 1, wherein the device is a mandibular advancement device (MAD), or an orthodontic device, or a device that repositions a mandible or a maxilla.
